(19) United States
(12) Patent Application Publication
(10) Pub. No.: US 2005/0048660 A1
(43) Pub. Date: Mar. 3, 2005

(54) Biomarker of maternal alcohol use during pregnancy

(75) Inventor: Cynthia F. Bearer, Cleveland Heights, OH (US)

Correspondence Address: TAROLLI, SUNDHEIM, COVELL TUMMINO & SZABO L.L.P., SUITE 1111, 526 SUPERIOR AVENUE, CLEVELAND, OH, 44114-1400, US

(73) Assignee: Case Western Reserve University

(21) Appl. No.: 10/653,744

(22) Filed: Sep. 2, 2003

(51) Int. Cl. G01N 33/92

(52) U.S. Cl. 436/132; 422/61

(57) ABSTRACT

A diagnostic test for determining maternal alcohol consumption during pregnancy comprises determining the level of at least one fatty acid ethyl ester in a bodily sample of a neonate and comparing the level of the at least one fatty acid ethyl ester in the bodily sample with at least one predetermined value.

### FIELD OF THE INVENTION

The present invention relates to a method of detecting maternal alcohol consumption during pregnancy and particularly to the detection of biological markers that can indicate maternal alcohol consumption during pregnancy.

### BACKGROUND

There has been increasing concern about the effects of ethanol on the developing fetus. Ethanol is known to cross the placenta and cause deleterious effects on the developing fetus. As little as 30 ml (1 ounce) of ethanol per day increases the risk of decreased birth weight. Even moderate drinking may be responsible for an increased risk of spontaneous abortion (See, S. E. Hyman and N. H. Cassem, “13. III. Alcoholism,” in D. C. Dale and D. D. Federman (eds.), Medicine, Scientific American, New York, page 11 (1995)); and Council on Scientific Affairs, J. Amer. Med. Assoc., 249:2517 (1983)).

The entire spectrum of prenatal alcohol damage, from mild to severe, is generally referred to as alcohol-related birth defects (ARBD). The frequency and severity of these anomalies appear to be dose-related and range from apparently clinically unaffected children to severely affected children suffering from fetal alcohol syndrome (FAS). Infants who have some, but not all of the physical characteristics of FAS are often referred to as exhibiting “Fetal Alcohol Effects” (FAE).

FAS is characterized by a distinctive facial appearance, prenatal onset growth deficiency, an increased frequency of developmental and mental retardation, and major congenital anomalies. Usually, FAS children suffer from central nervous system (CNS) deficiency (e.g., microcephaly and low IQ), slowness in growth, a characteristic group of facial abnormalities (e.g., short palpebral fissures, hypoplastic upper lip, and a short nose), and a variable array of major and minor malformations. Additional congenital abnormalities associated with FAS include cleft palate, cardiac malformations (especially atrial and ventricular septal defects), microphthalmia, hearing loss, joint anomalies, and a variety of dental and skeletal abnormalities. Neuropathologic examination often reveals significant abnormalities, neuronal migration occasionally associated with microcephaly, hydrocephaly, absence of the corpus callosum and cerebellar abnormalities. These infants commonly are short and of low birthweight. Often, they fail to thrive and do not grow as rapidly as other infants. In the newborn period, they are jittery or tremulous, a feature that is often confused with drug withdrawal symptoms. The pattern of wakefulness and sleep in affected newborns may also be disturbed. The neurologic abnormalities persist and in addition to developmental delay and mental retardation, such children are often poorly coordinated, tremulous and sometimes “hyperactive” in later life. In addition, children suffering from FAS have a greatly increased susceptibility to life-threatening, as well as minor infectious diseases, largely due to extensively impaired immune systems (See e.g., Johnson et al., Pediatr. Res., 15:908-911 (1981)). Although the exact mechanisms are not known, the effects of FAS may be due in part to the direct inhibition of embryonic cellular proliferation during early gestation by ethanol or acetaldehyde (Brown et al., Science 206:573-575 (1979)). However, there may also be selective fetal malnutrition due to placental injury (See Fisher and Karl, Recent Dev. Alcohol., 6:277-289 (1988)).

Drinking during pregnancy can also result in a spectrum of effects known as alcohol-related neurodevelopmental disorder (ARND), which range from severe cognitive and behavioral impairment without the classic facial dysmorphology to relatively subtle neurobehavioral deficits. It is estimated that 1% of all newborns are affected by prenatal ethanol exposure. School-age children whose mothers recalled having consumed more than 5 drinks on at least one occasion during pregnancy had learning problems, and 6.5-month-old infants whose mothers consumed 7 drinks per week on average had measurable deficits in performance on the Fagan Test of Infant Intelligence. The Centers for Disease Control and Prevention has reported an increase in the prevalence of binge drinking (≧5 drinks per occasion) among pregnant women from 0.7% in 1991 to 2.7% in 1999.

Diagnosis of FAS, FAE, and ARND is made clinically and based on a maternal history of alcohol consumption during pregnancy. Because this history is difficult to obtain, the true incidence of these conditions may be grossly underestimated. Some studies have shown a 100% failure to diagnose FAS or FAE. While the most severe case can be diagnosed at birth, in many cases the subtle signs of FAE may not become apparent until children reach school age. Learning difficulties and hyperactivity may be particularly troublesome to both children and parents if the source is not apparent.

The ability to recognize FAS varies according to the physician's skills and interests. While the diagnosis is easier with a known maternal history of alcohol abuse, there are many confounding variables such as reluctance to admit to and accurately report alcohol use, nutritional status, and other substance abuse, which make it difficult to interpret data regarding the relationship between the amount of alcohol consumed and FAS and FAE. Thus, FAS and FAE remain greatly under-diagnosed today, and early intervention is largely precluded.

Because alcohol is metabolized rapidly, there is currently no well-validated biological marker of exposure during pregnancy. Two studies have shown correlation of biological markers to maternal drinking and/or fetal outcome. In the first, hemoglobin-acetaldehyde adducts (HAA) and carbohydrate deficient transferrin (CDT) were not associated with the reported level of drinking in pregnant women with alcohol abuse. However, mean corpuscular volume (MCV) and gamma glutamyl transferase (GGT) were significantly higher in women drinking at least eight drinks per week compared with those drinking less than eight drinks per week. In another study, tests for CDT, GGT, MCH and HAA were combined. All women who reported drinking at least 14 drinks per week were positive for one or more markers. Having two or more positive markers was more predictive of infant outcome than any measure of self-reported drinking. Neither of these studies reported sensitivity or specificity of the biomarker. Thus, while statistically significant, the clinical usefulness of these tests is unclear.

A biological marker for risk levels of drinking during pregnancy would permit earlier identification and intervention for affected infants and would facilitate recognition of women at risk for drinking during their next pregnancy. It could also help improve understanding of the effects of prenatal alcohol exposure on neurobehavioral development. For example, it would provide a long-term, objective measure of fetal exposure that can supplement maternal recall, which is susceptible to social desirability and recall bias.

### SUMMARY OF THE INVENTION

The following presents a simplified summary of the invention in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

The present invention relates to a diagnostic test for determining maternal alcohol consumption during pregnancy. In the diagnostic test, the level of at least one fatty acid ethyl ester in a bodily sample of a neonate can be determined. The level of the at least one fatty acid ethyl ester in the bodily sample can then be compared with at least one predetermined value. The comparison can provide information for determining maternal alcohol consumption during pregnancy.

In one aspect of the invention, the predetermined value can comprise a single normalized value or a range of normalized values and can be based on fatty acid ethyl ester levels in comparable bodily samples from the general population or a select population of neonate subjects. In another aspect of the invention, the least one predetermined value can be a single value or a range of representative values and can be based on the fatty acid ethyl ester levels in the comparable bodily samples from the general population or a select population of neonate subjects. In yet another aspect of the present invention, the at least one predetermined value can be a plurality of fatty acid ethyl ester level ranges that are based on the fatty acid ethyl ester levels in comparable bodily samples from the general population or a select population of neonate subjects and the comparing step can comprise determining in which of the plurality of predetermined fatty acid ethyl ester level ranges the neonate's fatty acid ethyl ester level falls.

In another aspect, the at least one fatty acid ethyl esters can have the general formula CₙH₂ₙ₋ₓO₂, where n is an integer greater than 12 and x is selected from group consisting of 0, 1, 2, 3, and 4. Particularly, the at least one fatty acid ethyl ester can be selected from the group consisting of ethyl palmitate, ethyl oleate, and ethyl linoleate.

In a further aspect of the present invention, the level of the at least one fatty acid ethyl ester can be determined by isolating the at least one fatty acid ethyl ester from the bodily sample and detecting the isolated fatty acid ethyl ester by mass spectrometry. The at least one fatty acid ethyl ester can be isolated from the bodily sample by extracting the at least one fatty acid ethyl ester from the bodily sample and purifying the extracted fatty acid ethyl ester using chromatographic separation. A solvent can be used to extract the fatty acid ethyl ester from the bodily sample. The solvent can comprise a miscible solvent and/or an immiscible solvent. The miscible solvent can comprise water and acetone. The immiscible solvent can comprise hexane. In another aspect, the solvent can comprise a mixture of water, acetone, and hexane. In a further aspect, the chromatographic separation can be accomplished by applying hexane:ethyl acetate to the column.

In yet another aspect of the present invention, the level of the at least one fatty acid ethyl ester can be determined by a diagnostic assay. The diagnostic assay can include, for example, calorimetric assays and immunoassays. The immunoassays can comprise competitive immunoassays, immunometric assays, and immunosorbent assays.

The present invention also relates to a diagnostic test for characterizing a neonate's risk of developing or having fetal alcohol syndrome, fetal alcohol effects, and/or an alcohol-related neurodevelopmental disorder. In the diagnostic test, the level of the at least one fatty acid ethyl ester in a bodily sample of a neonate can be determined. The level of the at least one fatty acid ethyl ester in the bodily sample of the neonate can be compared with at least one predetermined value. The comparison provides information for characterizing the neonate's risk of developing or having fetal alcohol syndrome, fetal alcohol effects, and/or an alcohol-related neurodevelopmental disorder.

The predetermined value for characterizing the neonate's risk of developing or having fetal alcohol syndrome, fetal alcohol effects, and/or an alcohol-related neurodevelopmental disorder can comprise a single normalized value or a range of normalized values and can be based on fatty acid ethyl ester levels in comparable bodily samples from the general population or a select population of neonate subjects. In another aspect of the invention, the least one predetermined value can be a single value or a range of representative values and can be based on the fatty acid ethyl ester levels in the comparable bodily samples from the general population or a select population of neonate subjects. In yet another aspect of the present invention, the at least one predetermined value can be a plurality of fatty acid ethyl ester level ranges that are based on the fatty acid ethyl ester levels in comparable bodily samples from the general population or a select population of neonate subjects and the comparing step can comprise determining in which of the plurality of predetermined fatty acid ethyl ester level ranges the neonate's fatty acid ethyl ester level falls.

In another aspect, the at least one fatty acid ethyl ester can have the general formula CₙH₂ₙ₋ₓO₂, where n is an integer greater than 12 and x is selected from group consisting of 0, 1, 2, 3, and 4. Particularly, the at least one fatty acid ethyl ester can be selected from the group consisting of ethyl palmitate, ethyl oleate, and ethyl linoleate.

In a further aspect of the present invention, the level of the at least one fatty acid ethyl ester can be determined by isolating the at least one fatty acid ethyl ester from the bodily sample and detecting the isolated fatty acid ethyl ester by mass spectrometry. The at least one fatty acid ethyl ester can be isolated from the bodily sample by extracting the at least one fatty acid ethyl ester from the bodily sample and purifying the extracted fatty acid ethyl ester using chromatographic separation. A solvent can be used to extract the fatty acid ethyl ester from the bodily sample. The solvent can comprise a miscible solvent and/or an immiscible solvent. The miscible solvent can comprise water and acetone. The immiscible solvent can comprise hexane. In another aspect, the solvent can comprise a mixture of acetone and hexane. In a further aspect, the chromatographic separation can be accomplished by applying hexane:ethyl acetate to the column.

In yet another aspect of the present invention, the level of the at least one fatty acid ethyl ester can be determined by a diagnostic assay. The diagnostic assay can include, for example, colorimetric assays and immunoassays. The immunoassays can comprise competitive immunoassays, immunometric assays, and immunosorbent assays.

The present invention further relates to a kit comprising a means for at least partially isolating fatty acid ethyl esters from a bodily sample and an assay for determining the level of at least one of ethyl linoleate, ethyl palmitate, and/or ethyl oleate in the isolated fatty acid ethyl esters.

### BRIEF DESCRIPTION OF THE FIGURES

Further features of the present invention will become apparent to those skilled in the art to which the present invention relates from reading the following description of the invention with reference to the accompanying drawings in which:

FIG. 1 is a scattergram of absolute alcohol per drinking day averaged over pregnancy versus log ethyl oleate, μg/g (dry weight). 1 oz=2 standard drinks.

FIG. 2 is a receiver operating characteristic (ROC) curve assessing the sensitivity and specificity of ethyl oleate concentrations in meconium for identifying women who ingested at least 1.5 oz absolute alcohol per drinking day during pregnancy.

### DESCRIPTION OF THE INVENTION

The present invention provides diagnostic methods for determining maternal alcohol consumption during pregnancy as well as diagnostic methods for characterizing a neonate's risk of developing or having fetal alcohol syndrome, fetal alcohol effects, and/or an alcohol-related neurodevelopmental disorder. It has been found in accordance with the present invention that the levels of fatty acid ethyl esters in bodily samples obtained from neonates can be analyzed and quantified to determine maternal alcohol consumption during pregnancy as well as to characterize a neonate's risk of developing or having fetal alcohol syndrome, fetal alcohol effects, and/or an alcohol related neurodevelopmental disorder.

The present invention also relates to kits that comprise assays for at least one fatty acid ethyl ester. Such assays have appropriate sensitivity with respect to predetermined values selected on the basis of the present diagnostic methods and provide rapid and simple methods for the detection and quantification of fatty acid ethyl esters (e.g., ethyl palmitate, ethyl oleate, and/or ethyl linoleate) obtained from bodily samples of neonates.

The use of fatty acid ethyl esters as the basis to diagnose neonates is based on the recognition of the pathways of alcohol metabolism. In mammals, alcohol is metabolized primarily in the liver, via two different pathways, namely, the oxidation of ethanol to acetaldehyde by either alcohol dehydrogenase (ADH), or the microsomal ethanol-oxidizing system. Ethanol may be oxidized by alcohol dehydrogenase and the microsomal oxidizing system to generate acetaldehyde, which in turn is further oxidized to acetate by aldehyde dehydrogenase. Ethanol can also be metabolized in a non-oxidative fashion and esterified with a fatty acid to form fatty acid ethyl esters in a reaction catalyzed by numerous enzymes including triglyceride lipase and carboxylesterase. The enzyme activity is frequently referred to as fatty acid ethyl ester synthase.

Non-oxidative ethanol metabolism to produce fatty acid ethyl esters has been described and has been observed in both liver and extrahepatic tissues. In addition, the non-oxidative pathway has been described in fetal and placental tissues (See e.g., Bearer et al., Pediatr. Res., Vol. 31, 492-495 (1992)). The synthesis and accumulation of fatty acid ethyl esters following ethanol ingestion has been shown in human pancreas, liver, adipose tissue, heart, bone marrow, peripheral white blood cells, cerebral cortex, skeletal muscle, and the aorta. It is possible that this synthesis and accumulation of fatty acid ethyl esters may represent a mechanism for ethanol-induced damage or toxicity in organs lacking ADH. Indeed, the organs most frequently damaged by ethanol abuse have been shown to contain the highest levels of fatty acid ethyl ester synthase activity, and after acute intoxication, the highest level of fatty acid ethyl esters. Thus, the presence of fatty acid ethyl esters following ethanol ingestion, as well as the enzyme activity responsible for their synthesis provides circumstantial evidence that fatty acid ethyl esters are toxic metabolites, which may account in part for ethanol induced organ damage (Doyle et al., J. Lipid Res., 35:428-437 (1994)).

In the method of the present invention, a bodily sample can be obtained from a neonate whose mother is suspected of consuming alcohol during pregnancy. The bodily sample can be obtained either invasively or non-invasively from the neonate but is preferably obtained non-invasively. The bodily sample obtained from the neonate can potentially include breath, body fluids, such as urine, blood, cord blood, sputum, fecal matter, cerebrospinal fluid (CSF), semen, and saliva, as well as solid tissue, such as hair, skin, vernix, umbilical cord, and placenta. It will be appreciated by one skilled in the art that other bodily samples not listed can also be used in accordance with the present invention. In accordance with a preferred aspect of the present invention, the bodily sample can comprise a meconium sample that can be obtained non-invasively from the diaper of a neonate.

Meconium refers to the waste products, which accumulate as the intestinal contents of fetuses during gestation. Meconium can be comprised of desquamated intestinal skin and epithelial cells, pancreatic and intestinal secretions, and residue of swallowed amniotic fluid. Meconium is continuously formed and stored in the fetal intestines from late in the first trimester of gestation, i.e., from between 12-16 weeks, until birth. Unlike urine, which is excreted from the fetus in utero, meconium is not excreted until after birth. Meconium forms the first several excreta of newborn infant until a change to transitional milk stools is observed. Meconium tissue may therefore act as a biological time-capsule, in that each infant's meconium may reveal the history of the fetus in utero in terms of its exposure to various chemicals which become bound in the meconium matrix when meconium is formed.

The bodily samples can be obtained from the neonate using sampling devices such as swabs, syringes, or other sampling devices used to obtain liquid and/or solid bodily samples either invasively (i.e., directly from the neonate) or non-invasively (e.g., from diaper of the neonate). These samples can then be stored in storage containers, such as falcon tubes (e.g., 15 ml, propylene, Becton-Dickinson). The storage containers used to contain the collected sample can comprise a non-surface reactive material, such as polypropylene. The storage containers should generally not be made from untreated glass or other sample reactive material to prevent the sample from becoming absorbed or adsorbed by surfaces of the glass container.

Collected samples stored in the container may be stored under refrigeration temperature. For longer storage times, it is desirable that the collected sample be frozen to retard decomposition and facilitate storage. For example, samples obtained from the subject neonate can be stored in a falcon tube and cooled to a temperature of about −70° C.

In one aspect of the invention, a plurality of meconium excreta or bowl movements can be collected from a neonate's diaper and transferred to storage containers. Meconium has a very characteristic bluish-gray-green color and a rubber elastic texture, which differs greatly from the first transitional milk stool, which is characterized by being loosely formed and bright yellow in color. Meconium can be collected from the neonate suspected of gestational exposure by collecting and pooling each meconium sample produced from birth until the first appearance of transitional or milk stool appears.

Each meconium bowel movement for the subject neonate can be collected and stored in a storage container (e.g., falcon tube) having a volume capable of receiving all meconium samples produced by the subject neonate, typically from about 2.5 to about 5.0 grams. Meconium generally has an apparent density such that about one teaspoon of meconium approximately weighs about one gram. It is desirable to obtain at least about 1.0 grams of meconium from the neonate for analysis. The samples can then be frozen (e.g., −70° C.) until analyzed.

It will be appreciated that other bodily samples as well as methods of obtaining and storing bodily samples from neonates are well known and can be used by one skilled in the art.

The bodily sample obtained from the neonate can be analyzed to determine the level of at least one fatty acid ethyl ester in the bodily sample. The term “fatty acid ethyl ester” or “FAEE” as used in accordance with the present invention refers to compounds produced by the reaction of ethanol with free fatty acids. It is intended that the term not be limited to any particular compound nor is it related to any particular method of production, although in one embodiment, fatty acid ethyl esters can be produced by enzymatic conjugation. The term “fatty acid” refers to monobasic aliphatic acid containing only carbon, hydrogen, and oxygen, and consists of an alkyl radical attached to the carboxyl group. The saturated fatty acids have the general formula CₙH₂ₙO₂. The unsaturated fatty acids can contain one or more double bonds (e.g., two double bonds, three double bonds, and four double bonds). The term “ester” refers to organic compounds often formed by the combination of an acid and an alcohol, with elimination of water. The term “ethyl” refers to the group CH₃CH₂ (C₂H₅ or “Et—”).

In one aspect of the present invention, the bodily sample can be analyzed to determine the level of at least one fatty acid ethyl ester that has the general formula CₙH₂ₙ₋ₓO₂, where n is an integer greater than 12 and x is selected from the group consisting of 0, 1, 2, 3, and 4. Fatty acid ethyl esters having this general formula are readily separated and quantified using analytical techniques. In another aspect, the bodily sample can be analyzed to determine the level of at least one fatty acid ethyl ester selected from the group consisting of ethyl palmitate, ethyl oleate, and/or ethyl linoleate. Ethyl palmitate, ethyl oleate, and ethyl linoleate were found to be the most prevalent fatty acid ethyl esters in samples of meconium obtained from neonates.

It will be appreciated by one skilled in the art, that the level of at least one fatty acid ethyl ester can also comprise the sum of the levels of individual fatty acid ethyl esters. The sum of the levels of fatty acid ethyl esters can include all the fatty acid ethyl esters that are in the bodily sample or the sum of select fatty acid ethyl esters that are in the bodily sample. For example, the total level (i.e., sum) of substantially all the fatty acid ethyl esters can be determined. Likewise, the sum of the level of select fatty acid ethyl esters, such as ethyl palmitate, ethyl oleate, and/or ethyl linoleate can also be determined.

The level of the at least one fatty acid ethyl ester in the bodily sample can be determined by separating the at least one fatty acid ethyl ester from the bodily sample and then quantifying the amount of the at least one fatty acid ethyl ester in the separated sample. In one aspect of the invention, the at least one fatty acid ethyl ester can be separated from the bodily sample by contacting the bodily sample with at least one solvent under conditions such that an extracted sample is produced. The solvent can include any chemical useful for the removal (i.e., extraction) of the fatty acid ethyl ester from a solid mixture. For example, where the bodily sample comprises meconium, the solvent can include at least one of hexane, acetone, water, isooctane, or ethyl acetate. It will be appreciated by one skilled in the art that the solvent is not strictly limited to this context, as the solvent may be used for the removal of fatty acid ethyl esters from a liquid mixture, with which the liquid is immiscible in the solvent. Those skilled in the art will further understand and appreciate other appropriate solvents that can be employed to extract the fatty acid ethyl ester from the bodily sample.

The solvent can include solvent mixtures comprising miscible, partially miscible, and/or immiscible solvents. For example, the solvent can comprise a mixture of water, acetone, and hexane or a mixture of hexane and ethyl acetate. The solvent can also be combined with other solvents or liquids, which are not useful for the removal of the fatty acid ethyl ester. The other solvents in the solvent mixture can act as carriers, which facilitate mixing of the solvent with the bodily sample or transfer of the extracted fatty acid ethyl ester from the bodily sample.

In another aspect of the present invention, fatty acid ethyl esters can be extracted from meconium by contacting a sample of meconium obtained from a neonate with water and a water-miscible solvent (e.g., acetone). The fatty acid ethyl esters can then be separated from the acetone through the use of a solvent that is immiscible in water (e.g., hexane).

Following extraction of the bodily sample, the extracted sample can be extracted (i.e., purified) by, for example, solid phase extraction. One example of solid phase extraction uses column chromatography to purify the extracted sample. In column chromatography, a portion of a sample, dissolved in a mobile phase, is introduced at the head of a column packed with a stationary phase. The components of the sample distribute themselves between separate phases with some of the components remaining in the mobile phase and passing through the column and other of the components remaining in the stationary phase and being retained in the column. The components of the sample retained by the stationary phase (i.e., the eluate) can be eluted from the column by the introduction of an eluent to the column, which has a higher retention for the eluate than the stationary phase.

The column used in accordance with the present invention can comprise various packing materials, which are used to form the stationary phase. These packing materials can include, for example, silica gel, C18, C8, C2, cyclohexyl and phenyl bonded phases, XAD-2 resins, florisil, amino, cyano, diol, and alumina packings, ion exchange resins and any other known packing material that can be used to purify fatty acid ethyl esters. In one example of the present invention, the column comprises a silica gel (EM Science CAS 63231-67-4) that is washed with methanol and isooctane, degassed with nitrogen gas, and packed into a pasteur pipette.

It is not intended that the type of column be limited to a particular format. For example, it is contemplated that the commercially available materials such as those provided by Alltech can be used in the present invention, including but not limited to Adsorbosil, Adsorbosphere, Alltima, Econosil, Econosphere, hydroxyethyl methacrylate polymer (HEMA), macrosphere 300, C18/anion, C18/cation, Tenax-TA, Tenax-GC, “amine packings” (e.g., column materials commercially available from suppliers such as Alltech, including, Alltech Amine Packing, Carbowax™, Carbowax™20M, Porapak™, HayeSep™, Chromosorb™, Amipack™., Apiezon™, and AT™-WAX), DB™-WAX, Superox™II, HP-20M, Supelcowax™-10, and Versapack materials. It is not intended that the column packing materials be limited to a specific supplier or composition.

The mobile phase used to introduce the at least one fatty acid ethyl ester into the column can comprise any solvent or combination of solvents that is capable of dissolving the fatty acid ethyl ester and has a lower retention for the at least one fatty acid ethyl ester than the packing material. For example, where the packing material comprises silica gel the solvent can comprise hexane. The eluent used to elute the eluate from the column can include any solvent or combination of solvent that has a higher retention for the at least one fatty acid ethyl ester than the packing material. For example, where the packing material comprises silica gel, and the mobile phase comprises hexane, the eluant can comprise a mixture of hexane and ethyl acetate (e.g., 100:1).

It will be appreciated to one skilled in the art that other solvents and eluents can be used in accordance with the present invention. It will also be appreciated by one skilled in the art that other extraction techniques can be used to separate the at least one fatty acid ethyl ester from the bodily sample and/or to purify an extracted sample. These extraction techniques can include, for example, other types of solid phase extraction (SPE), solid phase membrane extraction (SPME), supercritical fluid extraction (SFE), and immunoaffinity extraction techniques. It will be appreciated that still other extraction techniques or methods are known and can potentially be used in accordance with the present invention.

It will also be appreciated that other types of chromatographic techniques can be used in place of or in combination with the extraction techniques to separate the at least one fatty acid ethyl ester from the bodily sample. For example, the fatty acid ethyl ester can be separated from the extracted sample by thin layer chromatography as disclosed in E. Mac et al., Pediatr. Res., 35:238A (1994), gas chromatography, high pressure liquid chromatography. Additionally, other non-chromatographic methods can be used to purify the at least one fatty acid ethyl ester from the extracted sample.

Once separated, the fatty acid ethyl ester can be quantified to determine the amount (i.e., the level) of the at least one fatty acid ethyl ester in the bodily sample. Quantification can be performed using analytical techniques, such as gas chromatography (GC), high pressure liquid chromatography (HPLC), capillary electrophoresis (CZE), gas chromatograph flame ionization detection (GC-FID), and mass spectrometry (MS). A preferred analytic technique for quantifying the amount of fatty acid ethyl ester in the purified sample includes tandem mass spectrometry (MS/MS). A tandem mass spectrometer can be thought of as two mass spectrometers in series connected by a chamber that can break a molecule into pieces. This chamber is known as a collision cell. A sample is sorted and weighed in the first mass spectrometer, then broken into pieces in the collision cell, and the piece or pieces sorted and weighed in the second mass spectrometer.

Prior to analysis, an internal standard can be added to the sample extract for quantification of the fatty acid ethyl ester. For analysis by mass spectrometry, the internal standard can include 13C-labeled analogues of the fatty acid ethyl esters to be determined. These internal standards are preferred because they correct for instrument variation and allow for more accurate quantification.

In another aspect of the invention, a chromatographic instrument, such as a gas chromatograph, can be coupled to the tandem mass spectrometer to form, for example a gas chromatograph tandem mass spectrometer GC/MS/MS. Coupling a chromatographic instrument to the tandem mass spectrometer can facilitate placement of the fatty acid ethyl ester before the mass spectrometer. It will be appreciated that other instruments can be coupled to a tandem mass spectrometer or mass spectrometer. Such other instruments can include, for example, a high pressure liquid chromatograph.

The use of tandem mass spectrometry and, in particular, gas chromatography tandem mass spectrometry, in the quantification of the at least one fatty acid ethyl ester is advantageous because tandem mass spectrometry measures many different molecules in a single test. Conventional mass spectrometers and other analytical instruments require the use of several tests to look at different types of molecules, which is more time consuming and, hence, more expensive. With tandem mass spectrometry the results are available more quickly and are more accurate. For example, at a specificity of 83%, the sensitivity provided by tandem mass spectrometry was 84% for the detection of ethyl oleate in meconium samples, compared with only 70% to detect heavy drinking (14 drinks/week prior to pregnancy) using GC-FID. At a specificity of 100%, the sensitivity provided by GC/MS/MS was 68%, compared to 52% using GC-FID.

It will be appreciated by one skilled in the art that in addition to instrumental screening and quantitative techniques to determine the level of the at least one fatty acid ethyl ester in the bodily sample other methodologies can be used. These other methodologies can include, for example, a calorimetric assay where a chromophore that serves as a substrate for the at least one fatty acid ethyl ester generates a product with a characteristic wavelength which may be followed by any of various spectroscopic methods including UV-visible or fluorescence detection. Additional details of calorimetric based assays can be found in Kettle, A. J. and Winterbourn, C. C. (1994) Methods of Enzymology. 233: 502-512; and Klebanoff, S. J., Waltersdorph, A. N. and Rosen, H. (1984) Methods in Enzymology. 105: 399-403, both of which are incorporated herein by reference.

Other methods for determining the level of the at least one fatty acid ethyl ester include immunoassay methods. In these methods, an immunoassay system is provided that includes a specific antibody to an antigen (i.e., the at least one fatty acid ethyl ester) and a system to measure the antigen in the bodily sample. The procedures for antibody production are standard in the art and will be readily conducted by persons of ordinary skill in the art. For example, antibodies can be raised to the at least one fatty acid ethyl ester itself or using the compound, such as a hapten conjugated to an acceptable carrier, where the carrier is an immunogen. Antibodies, such as monoclonal antibodies, can be obtained from continuous cell lines. Conventional techniques for producing monoclonal antibodies are the hybridoma technique of Kohler and Millstein (Nature 356:495-497(1975)) and the human B-cell hybridoma technique of Kosbor et al. (Immunology Today 4:72 (1983)). Such antibodies may be of any immunoglobulin class including IgG, IgM, IgE, IgA, IgD, and any class thereof.

Examples of immunoassay systems that can be used in accordance with the present invention include competitive immunoassays systems (such as radioimmunoassays (RIA)), immunometric assays, and immunosorbent assays, such as an enzyme linked immunosorbent assay (ELISA). In competitive immunoassays, an antibody to a specific antigen (i.e., the at least one fatty acid ethyl ester) is provided. The antibody at a very specific and defined limited concentration, binds the antigen in the sample, and an antigen labeled with some detection system, such as alkaline phosphatase. The amount of either the bound or free labeled antigen added to the reaction is measured at the end of the immunological binding reaction and the percentage bound is inversely proportional to the amount of unlabeled antigen in either the standard or the samples. The separation at the end of the immunological binding reaction can be by a number of separation systems using, for example, a microtiter plate, paramagnetic beads, or dextran-coated charcoal.

Immunometric assays (i.e., sandwich assays) use two or more antibodies to sandwich the antigen. Typically, one of the antibodies is bound to a separation system (i.e., solid phase antibody), such as a microtiter plate, and one antibody is used to detect the antigen (ie., bound to a detection enzyme, such as alkaline phosphatase). Typically, the amount of solid phase antibody and detection antibody are in large excess over the amount of antigen in the sample. This forces the kinetics of the binding of the antigen to the solid phase, and detection antibody conjugate to the antigen to be pseudo-first order. The result is an assay that produces a signal that is proportional to the amount of antigen in solution.

Immunosorbant assays, such as ELISA, use a solid phase coated with an antibody to the antigen (i.e., the at least one fatty acid ethyl ester). A bodily sample containing the antigen is applied to the solid phase. A second antibody with a detection system, such as a detection enzyme, is bound to the antigen and the presence of the detection enzyme is detected to determine the amount of antigen.

The detection method for immunoassays used in accordance with the present invention can include radioactivity, colorimetry, fluorescence, and chemiluminescence. To detect lower concentrations or to obtain faster results, it is desirable that chemiluminescence detection be used. Chemiluminescence detection can preferably be used with immunosorbent assays, such as ELISA. Luminescence from this assay can be measured using a commercially available luminometer, such as a Packard Lumicount microplate luminometer. This luminometer provides a sensitive, high throughput, and economical alternative to conventional colorimetric activities. It will be appreciated by one skilled in the art that other detection methods can also be used with the immunoassays in accordance with the present invention.

Once the level of the at least one fatty acid ethyl ester is determined, the level of the at least one fatty acid ethyl ester in the neonate's bodily sample can be compared to a predetermined value to provide information for determining the maternal alcohol consumption during pregnancy. The predetermined value can be based upon the level the at least one fatty acid ethyl ester in comparable samples obtained from the general neonate population or from a select population of neonate subjects. For example, the select population may be comprised of apparently healthy neonate subjects. “Apparently healthy”, as used herein, means (1) neonates whose mothers for religious, ethnic, and/or other reasons have historically abstained from the consumption of alcohol during pregnancy, (2) neonates whose mothers have indicated by questionnaire and/or some other survey method that they have abstained from the consumption of alcohol during pregnancy, and (3) neonates who at a time remote from birth (e.g., about 10 to about 12 years) have not demonstrated any signs or symptoms indicating the presence of disease, such as alcohol-related developmental disorder, fetal alcohol effects, and/or fetal alcohol syndrome (i.e., children if examined by a medical professional, would be characterized as healthy and free of symptoms of disease, such as alcohol-related developmental disorder, fetal alcohol).

The predetermined value can be related to the value used to characterize the level of the at least one fatty acid ethyl ester in the bodily sample obtained from the test subject. Thus, if the level of the at least one fatty acid ethyl ester is an absolute value, such as the mass (e.g., grams) of the at least one fatty acid ethyl ester per gram of meconium sample, the predetermined value can also be based upon the mass (e.g., grams) of the at least one fatty acid ethyl ester in neonates in the general population or a select population of human subjects. Similarly, if the level of the at least one fatty acid ethyl ester is a representative value such as an arbitrary unit, the predetermined value can also be based on the representative value.

The predetermined value can take a variety of forms. The predetermined value can be a single cut-off value, such as a median or mean. The predetermined value can be established based upon comparative groups such as where the level of the at least one fatty acid ethyl ester in one defined group is double the level of the at least one fatty acid ethyl ester in another defined group. The predetermined value can be a range, for example, where the general neonate population is divided equally (or unequally) into groups, or into quadrants, the lowest quadrant being neonates with the lowest levels of the at least one fatty acid ethyl ester, the highest quadrant being individuals with the highest levels of the at least one fatty acid ethyl ester.

The predetermined value can be derived by determining the level of the at least one fatty acid ethyl ester in the general neonate population. Alternatively, the predetermined value can be derived by determining the level of the at least one fatty acid ethyl ester in a select population. For example, an apparently healthy neonate population may have a different normal range of at least one fatty acid ethyl ester than a different ethnic or geographically located population based on the diet of such population. Accordingly, the predetermined values selected may take into account the category in which an neonate falls. Appropriate ranges and categories can be selected with no more than routine experimentation by those of ordinary skill in the art.

Predetermined values of the at least one fatty acid ethyl ester, such as for example, mean levels, median levels, or “cut-off” levels, are established by assaying a large sample of neonates in the general population or the select population and using a statistical model such as the predictive value method for selecting a positivity criterion or receiver operator characteristic curve that defines optimum specificity (highest true negative rate) and sensitivity (highest true positive rate) as described in Knapp, R. G., and Miller, M. C. (1992). Clinical Epidemiology and Biostatistics. William and Wilkins, Harual Publishing Co. Malvern, Pa., which is specifically incorporated herein by reference. A “cutoff” value can be determined for each fatty acid ethyl ester that is assayed.

Alternatively, the level of the at least one fatty acid ethyl ester can be compared to a predetermined value to provide a risk value which characterizes the neonate's risk of developing fetal alcohol syndrome, fetal alcohol effects, and/or an alcohol-related neurodevelopmental disorder.

The levels of at least one fatty acid ethyl ester, in the neonate's bodily sample may be compared to a single predetermined value or to a range of predetermined values. If the level of the present risk predictor in the test subject's bodily sample is greater than the predetermined value or range of predetermined values, the test subject is at greater risk of developing fetal alcohol syndrome, fetal alcohol effects, and/or an alcohol-related neurodevelopmental disorder than neonates with levels comparable to or below the predetermined value or predetermined range of values. In contrast, if the level of the present risk predictor in the test subject's bodily sample is below the predetermined value or range of predetermined values, the test subject is at a lower risk of developing fetal alcohol syndrome, fetal alcohol effects, and/or an alcohol-related neurodevelopmental disorder than neonates with levels comparable to or above the predetermined value or range of predetermined values. For example, a test subject who has a higher level of fatty acid ethyl ester, such as ethyl oleate, ethyl linoleate, and/or ethyl palmitate as compared to the predetermined value is at high risk of developing fetal alcohol syndrome, fetal alcohol effects, and/or an alcohol-related neurodevelopmental disorder, and a test subject who has a lower number of fatty acid ethyl esters, such as ethyl oleate, ethyl palmitate, and/or ethyl linoleate compared to the predetermined value is at low risk of developing fetal alcohol syndrome, fetal alcohol effects, and/or an alcohol-related neurodevelopmental disorder. The extent of the difference between the test subject's risk predictor levels and predetermined value is also useful for characterizing the extent of the risk and thereby, determining which neonates would most greatly benefit from certain aggressive therapies. In those cases, wherein the predetermined value ranges are divided into a plurality of groups, such as the predetermined value ranges for neonates at high risk, average risk, and low risk, the comparison involves determining into which group the test subject's level of the relevant risk predictor falls.

The present diagnostic tests are useful for determining if and when therapeutic agents which are targeted at treating fetal alcohol syndrome and/or an alcohol-related neurodevelopmental disorder should and should not be prescribed for a neonate. For example, neonates with values of a fatty acid ethyl ester, such as ethyl palmitate, ethyl oleate, and ethyl linoleate, above a certain cutoff value, or that are in the higher tertile or quartile of a “normal range,” could be identified as those in need of more aggressive intervention with therapeutic or surgical intervention.

One of the most attractive findings of increased levels fatty acid ethyl esters, such as ethyl palmitate, ethyl oleate, and/or ethyl linoleate as a predictor of risk for developing fetal alcohol syndrome and/or an alcohol-related neurodevelopmental disorder is that it represents an independent marker. Thus, the present diagnostic tests are especially useful to identify neonates at increased risk who might otherwise not have been identified by existing screening protocols/methods. Moreover, the present risk predictors can be used in combination with currently used risk predictors, such as maternal surveys and algorithms based thereon to more accurately characterize a neonate's risk of developing fetal alcohol syndrome and/or an alcohol-related neurodevelopmental disorder.

### EXAMPLES

The following examples serve to illustrate certain preferred embodiments and aspects of the present invention and are not to be construed as limiting the scope thereof. Unless otherwise indicated, the solvents and alcohols used in these Examples were obtained from Fisher. Also, unless otherwise indicated the solvents and alcohols used herein were HPLC grade.

### Example 1

Preparation of Silica Columns

In this Example, the silica gel was prepared by placing one volume of silica gel (EM Science CAS 63231-67-4) into a glass beaker. The silica gel was washed with HPLC grade methanol (Fisher), the gel allowed to settle, and the methanol decanted. This methanol wash was repeated three times. The gel was allowed to dry overnight in a fume hood. The silica gel was then resuspended in HPLC grade isooctane (Fisher), and placed in a side arm flask. The openings of the sidearm and flask were covered with aluminum foil, and stored at room temperature until use.

The columns were prepared by first washing 5″ borosilicate Pasteur pipettes in HPLC grade acetone, and allowed to dry. A small pellet of glass wool was then packed into the barrel of each pipette. The silica gel, prepared as described above, was degassed with nitrogen gas, at 30° C., for five minutes in an evaporator. After degassing, the gel was gently swirled, to produce a slurry. This slurry was then poured into the glass wool-packed Pasteur pipettes, until the height of the packed silica gel was approximately 5 cm., and packed in the barrel of the pipettes. The packed material in each pipette was then washed five times with 1 ml aliquots of hexane.

### Example 2

FAEE Extraction from Meconium

In this Example, FAEEs were extracted from meconium. First, a water-miscible solvent, acetone, was used to extract the meconium. Then, the FAEEs were separated from the acetone through use of a solvent that is immiscible in water, hexane.

In this Example, mixed standards were prepared by adding 100 μl of mixed ethyl esters and 100 μl of 1 mM 17:0 heptadecanoic ethyl ester internal standard into GC vials fitted with glass inserts, capped tightly, and vortexed for one minute at a speed setting of 3-4. The vortexing was accomplished so as to create full swirling.

One gram of the meconium sample obtained from a healthy neonate was added to a 30 ml Corex tube that had been pre-washed with acetone. One ml of distilled water was added to the Corex tube. The internal standard (100 μl of 1 mM 17:0 heptadecanoic ethyl ester) was added to the sample. The tube was thoroughly vortexed for approximately one minute at a speed setting of 3-4, again to create full swirling.

Three ml of acetone was added to the meconium sample in the Corex tube. The meconium was mixed into the acetone with a spatula until it became dry and fibrous. The spatula was rinsed using 2 ml of acetone and the rinse placed in the tube. The tube was vortexed for one minute at a speed setting of 3-4, after which 5 ml of hexane was added. The tubes were then vortexed for 1 minute at a speed setting of 1-2, and then centrifuged for 5 minutes at 800×g.

The top layer, containing the hexane phase was transferred to a clean, acetone-washed Corex tube and stored at room temperature. An additional 5 five ml of hexane were added to the meconium/acetone/water mixture, the tube was vortexed for 1 minute at a speed setting of 1-2, and then centrifuged for 5 minutes at 800×g. The top layer (i.e., the hexane phase) was added to the Corex tube containing the first hexane phase collected. The bottom, aqueous layer was discarded.

The hexane phase was dried under nitrogen gas, at 30° C., until complete dryness. The dried sample was then resuspended in 1 ml hexane, vortexed for 15 seconds at a speed setting of 1-2, and added to the silica gel column prepared according to the method of Example 1. The flow-through from the silica gel column was collected in a test tube. The column was then washed four times with 1 ml hexane. The wash was collected in the same test tube as the flow through, and saved until the GC results were analyzed to show that FAEEs did not pass through the column during the wash steps.

A fresh solution of hexane:ethyl acetate 160:1 was then prepared. Four 1 ml aliquots were added to the column, and the eluant was collected in one test tube and stored.

A fresh solution of hexane and ethyl acetate (100:1) was then prepared. This solution was then used to elute the FAEEs from the column. This elution was conducted five times with 1 ml aliquots. The total 5 ml eluate was collected in a clean test tube. If any of this 100:1 eluate dripped on the side of the collection tube, the tube sides were washed with 0.4 ml hexane and the wash collected into a tube. If any silica gel was present in the eluate, the purification was repeated by running all of the washes through a new silica gel column.

The eluate (5 ml) was dried down under nitrogen gas until complete dryness. The sample was resuspended with 200 μl hexane, ensuring that the sides of the test tube were rinsed. The sample was then vortexed for 15 seconds at a speed setting of 1. The sample was transferred to a GC vial and tightly capped. These steps were determined to be time-sensitive, due to the possibility of hexane evaporation. If hexane evaporation occurs, higher concentrations than expected will result. Thus, it is desirable that the steps are quickly and consistently performed for each sample, with one sample being processed at a time (i.e., all of the samples should not be resuspended and transferred at once).

The GC was loaded with the sample. For each run, the GC was loaded with a hexane standard, a mixed FAEE standard, and a 17:0 FAEE standard, as well as the samples. FAEE were clearly identified in some, but not all meconium samples. Nonetheless, recovery of the internal standard from the meconium was >90%, for all samples.

### Example 3

Correlation of FAEE in Meconium and Maternal Alcohol Use

In this Example, the correlation between FAEE in meconium and maternal alcohol use was investigated.

Methods:

Participants:

Mothers were recruited during pregnancy at an outpatient clinic that serves a predominantly Cape Coloured (mixed race) population to participate in a prospective study on the effects of heavy prenatal alcohol exposure on cognitive and behavioral development. The Cape Coloured are descendents of European, Malaysian, Khoi (Hottentot) aboriginal, and black African ancestors. Very heavy alcohol use is unusually prevalent among women in this community due to their very poor psychosocial and economic circumstances and the historically prevalent but now outlawed practice of paying Coloured workers on wine-producing grape and fruit farms with wine. The data is based on the first 27 infants in this cohort for whom meconium samples were collected.

Each gravida was interviewed about her alcohol consumption at recruitment, using an interview derived from the time-line follow back approach developed by Sokol, Martier, and Ernhart (1983)(18) (see Jacobson, S. et al., 2002). Antenatal care was initiated at a median gestational age of 19 weeks (range=6-34); median gestational age at recruitment was 25 weeks (range=8-37). Any woman who reported an average of at least 1.0 oz absolute alcohol (the equivalent of two standard drinks) per day or at least two incidents of binge drinking (≧5 standard drinks) per month during the first trimester of pregnancy was invited to participate in the study. The next woman initiating antenatal care whose gestational age was within 2 weeks of the heavy drinking mother was also invited to participate in the study, provided that she drank less than seven drinks per week (0.5 oz AA per day) and did not binge drink. Women less than 18 years of age and those with diabetes, epilepsy, or cardiac problems requiring treatment were not invited to participate. Religiously-observant Muslim women were also excluded because their religious practices prohibit alcohol consumption. Infant exclusionary criteria were major chromosomal anomalies, neural tube defects, multiple births, and seizures.

Informed consent was obtained from each mother at the time of recruitment in accordance with the institutional review boards of the University of Witwaterstrand, Wayne State University, Case Western Reserve University, and the Centers for Disease Control and Prevention and in accordance with the Helsinki Declaration of 1975, as revised in 1983. Once the unusually high rates of alcoholism and FAS became evident, a home visitor intervention based on principles of motivational interviewing was implemented in collaboration with the Parent Centre, a community-based parenting program run by Stephen Rollnick, Ph.D, and Mireille Landman, M.A. Arrangements were made to refer mothers to an alcohol treatment facility affiliated with the Department of Obstetrics, University of Cape Town School of Medicine.

Procedure:

Each mother was interviewed in Afrikaans regarding her alcohol and drug use at recruitment, at a follow-up antenatal visit, and when the infant was 1 month old. During recruitment, the mother was asked about her drinking on a day-by-day basis during a typical 2-week period around the time of conception, with recall linked to specific times of day and activities. She was then asked whether she continued her usual drinking pattern after becoming pregnant and, if not, when her drinking had changed and what she drank on a day-by-day basis during the past 2 weeks. At the follow-up antenatal visit, the mother was again asked about her drinking during the previous 2 weeks. If there were any weeks since the recruitment visit when she drank greater quantities, she was asked to report her drinking for those weeks as well. At the 1-month postpartum visit, the mother was asked about her drinking during a typical 2-week period during the latter part of pregnancy, as well as her drinking during any weeks during that period when she drank greater quantities. Volume was recorded for each type of alcohol beverage consumed each day and converted to oz of absolute alcohol (M) using multipliers proposed by Bowman et al. (1975)(21) (liquor-0.4, beer-0.04, wine-0.2). Three summary measures were constructed for each of five time periods: conception, first, second, and third trimester, and an overall average for the entire pregnancy. The summary measures of alcohol intake were overall average oz AA/day, oz AA per drinking day (quantity per occasion), and frequency (drinking days/week).

Random meconium samples were scraped from the infant's diaper into falcon tubes (15 ml, polypropylene, Becton-Dickinson) within 24 hours following delivery. The samples were refrigerated immediately or within a few hours and then frozen. Most (89%) were frozen at −19C within 1 day after collection; three were frozen after 2-10 days. Toward the end of the data collection, all specimens were transferred to a −70C freezer and maintained at −70C until analysis. The length of time between collection and being frozen at −70C ranged from 0 days to 13.4 months (interquartile range=14-83 days). When the FAEE concentrations in the three meconium specimens that were frozen at −19C more than 1 day after collection were compared with those that were frozen immediately, the mean level of one FAEE was lower but two others were higher, indicating that there was no general tendency for the FAEEs to deteriorate over several days of refrigeration. None of the FAEE concentrations were related to the length of time that passed between collection and storage at −70C (all p's >0.25).

Meconium Analysis:

Isolation of FAEEs from meconium was performed as previously described in Example 1 and Example 2. The dry weight of meconium was obtained by drying the meconium left in the water phase after the FAEE were extracted using a speedvac. Samples of isolated FAEEs were initially analyzed by gas chromatography/flame ionization detection, which showed that the predominant FAEEs in meconium are ethyl palmitate, ethyl oleate and ethyl linoleate. Samples were subsequently analyzed for these three FAEEs by GC/MS/MS using a TSQ 7000 mass spectrometer (San Jose, Calif.) interfaced to a Trace GC using methane chemical ionization and low energy collisions with argon. Prior to analysis, ¹³C-labeled analogues of the three FAEEs were added to the sample extracts to correct for instrument variation and to allow a more accurate quantitation. The two most prevalent fragments of ethyl palmitate, ethyl oleate and ethyl linoleate were quantitated.

Statistical Analysis:

Pearson correlation analyses were used to examine the relation of each of the FAEEs with three self-reported measures of alcohol consumption during pregnancy, as well as the relation of ethyl oleate to alcohol ingested per occasion at conception and during each trimester of pregnancy. A receiver operating characteristics (ROC) curve was constructed to assess the efficiency of meconium ethyl oleate concentration in identifying mothers who drank at least 1.5 oz AA per occasion during pregnancy.

Results:

The characteristics of the sample are shown in Table 1.

TABLE 1
SAMPLE CHARACTERISTICS

| | N | Mean or % | SD | Range |
|---|---|---|---|---|
| Maternal | | | | |
| Age at delivery | 27 | 26.13 | 6.51 | 17.78-43.82 |
| Education (years) | 27 | 8.59 | 2.71 | 0.00-12.00 |
| Marital Status | 27 | 48.15 | — | — |
| (% married) | | | | |
| Parity | 27 | 1.33 | 1.78 | 0-12.00 |
| Infant | | | | |
| Gestational age | 27 | 39.73 | 1.77 | 36.43-43.00 |
| (wk) | | | | |
| Birth weight (g) | 27 | 2967.22 | 596.44 | 1500.00-4240.00 |
| Head circumference | 27 | 32.81 | 1.92 | 29.00-36.00 |
| (cm) | | | | |
| Gender (% male) | 27 | 66.70 | — | — |
| Self-reported alcohol | | | | |
| consumption | | | | |
| Average oz AA^{a}/day | 27 | 0.86 | 1.48 | 0.00-7.36 |
| Average oz | 27 | 2.57 | 2.00 | 0.00-7.47 |
| AA^{a}/drinking day | | | | |
| Frequency (days/ | 27 | 1.34 | 1.48 | 0.00-6.65 |
| week) | | | | |
| FAEES^{b} of Alcohol | | | | |
| Ethyl palmitate | 26 | 6.30 | 16.53 | 0.02-62.26 |
| Ethyl oleate | 25 | 1.44 | 5.49 | 0.01-27.66 |
| Ethyl linoleate | 27 | 9.15 | 25.07 | 0.00-93.94 |

^{a}Absolute alcohol.
^{b}Fatty ethyl esters of alcohol, μg/g dry weight (most prevalent ion).

Seventeen of the women drank heavily during pregnancy (≧4 drinks per occasion; in most cases, 1-2 days/week), four drank heavily but less frequently (<3 days per month), and six abstained during pregnancy. All 27 samples of meconium contained ethyl linoleate, with 26 containing ethyl palmitate and 25 containing ethyl oleate. The most predominant FAEE was ethyl linoleate, with ethyl palmitate second, and ethyl oleate third.

GC/MS/MS analysis yielded two prevalent ions for each FAEE. Because the intercorrelations between these ions for each FAEE were exceptionally high (r's ranged from 0.99 to 1.00), subsequent analysis used only the first prevalent ion. The concentrations of these FAEEs in the meconium were highly correlated to each other (r's ranged from 0.61 to 0.86, median=0.79), particularly ethyl palmitate with ethyl oleate, both on a per wet weight and on a per dry weight basis.

To determine the measure of alcohol consumption most strongly correlated to FAEEs in meconium, FAEE concentrations were examined in relation to three measures averaged over pregnancy: average absolute ounces of alcohol per day, average ounces of absolute alcohol per drinking day, and number of drinking days per week (Table 2).

TABLE 2
CORRELATIONS OF SELF-REPORTED ALCOHOL
CONSUMPTION WITH CONCENTRATIONS OF
FATTY-ACID ETHYL ESTERS OF ALCOHOL
IN MECONIUM

| | | Average alcohol | Average alcohol | |
|---|---|---|---|---|
| Frequency | N | per day | per drinking day | (days/week) |
| Adjusted for | | | | |
| wet weight | | | | |
| Ethyl palmitate | 26 | .25 | .34† | .29 |
| Ethyl oleate | 25 | .34† | .48* | .32 |
| Ethyl linoleate | 27 | .26 | .27 | .29 |
| Adjusted for | | | | |
| dry weight | | | | |
| Ethyl palmitate | 26 | .20 | .35† | .24 |
| Ethyl oleate | 25 | .29† | .51** | .24 |
| Ethyl linoleate | 27 | .21 | .27 | .24 |

†p < 0.10,
*p_ < 0.05,
**p < 0.01.

As shown in Table 2, the highest correlations were found for average alcohol per drinking day (AADD) with ethyl oleate, whether measured on a per gram wet weight or dry weight basis. The rest of the analyses were, therefore, performed using ethyl oleate as the biomarker and AADD as the measure of drinking.

Since meconium does not begin formation until the second trimester, we predicted that maternal drinking in the second and third trimester would correlate most highly with the concentration of FAEEs in meconium. Correlations of ethyl oleate concentration to maternal self-reported AADD for the periods at conception, 1^{st} trimester, 2^{nd} trimester and 3^{rd} trimester were 0.29, 0.38, 0.52 (p<0.01), and 0.42 (p<0.05) respectively based on wet weight, and 0.32, 0.42 (p<0.05), 0.55 (p<0.01), and 0.40 (p<0.05) based on dry weight, as anticipated.

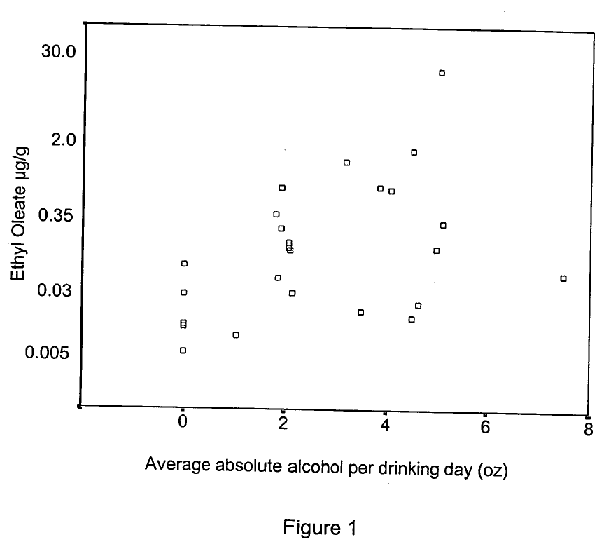

A scattergram of the relation of log ethyl oleate, μg/g dry weight, with absolute alcohol per drinking day averaged over pregnancy is shown in FIG. 1. The general trend is for ethyl oleate concentration to increase with increasing amounts of reported drinking. By observation, higher values of ethyl oleate were seen only at 1.5 ounces of absolute alcohol per drinking day or greater. Hence, further analyses were based on a level of 1.5 ounces of absolute alcohol per drinking day.

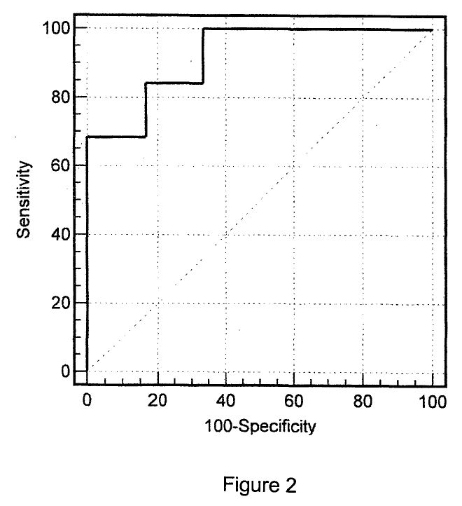

To assess the efficiency of the meconium test to identify women reporting drinking 1.5 ounces of absolute alcohol per drinking day or more, a receiver operating characteristics (ROC) curve was constructed as shown in FIG. 2. The area under the curve was 0.92, which was highly significantly different than 0.5 with 95% a confidence interval of 0.80 to 1.00.

The positive and negative predictive values of the meconium assay to identify women drinking 1.5 or more ounces of absolute alcohol per drinking day were calculated from the ROC curves and are shown in Table 3 below.

TABLE 3
RECEIVER OPERATOR
CHARACTERISTICS (ROC) CURVE FOR ETHYL OLEATE

| | | | Positive | Negative |
|---|---|---|---|---|
| | | | Predicted | Predicted |
| Cut-off^{a} | Sensitivity (%) | Specificity (%) | Value | Value |
| <0.006 | 100.00 | 0.0 | 0.76 | — |
| 0.006 | 100.00 | 16.7 | 0.79 | 1.00 |
| 0.009 | 100.00 | 33.3 | 0.83 | 1.00 |
| 0.012 | 100.00 | 50.0 | 0.86 | 1.00 |
| 0.013 | 100.00 | 66.7 | 0.91 | 1.00 |
| 0.016 | 94.7 | 66.7 | 0.90 | 0.80 |
| 0.020 | 89.5 | 66.7 | 0.90 | 0.67 |
| 0.025 | 84.2 | 66.7 | 0.89 | 0.57 |
| 0.032 | 84.2 | 83.3 | 0.94 | 0.63 |
| 0.034 | 78.9 | 83.3 | 0.94 | 0.56 |
| 0.052 | 73.7 | 83.3 | 0.93 | 0.50 |
| 0.061 | 68.4 | 83.3 | 0.93 | 0.46 |
| 0.077 | 68.4 | 100.0 | 1.00 | 0.50 |
| 0.122 | 63.2 | 100.0 | 1.00 | 0.46 |
| 0.131 | 57.9 | 100.0 | 1.00 | 0.43 |
| 0.132 | 52.6 | 100.0 | 1.00 | 0.40 |
| 0.155 | 47.4 | 100.0 | 1.00 | 0.38 |
| 0.233 | 42.1 | 100.0 | 1.00 | 0.35 |
| 0.283 | 36.8 | 100.0 | 1.00 | 0.33 |
| 0.349 | 31.6 | 100.0 | 1.00 | 0.32 |
| 0.750 | 26.3 | 100.0 | 1.00 | 0.30 |
| 0.788 | 21.1 | 100.0 | 1.00 | 0.29 |
| 0.812 | 15.8 | 100.0 | 1.00 | 0.27 |
| 1.778 | 10.5 | 100.0 | 1.00 | 0.26 |
| 2.468 | 5.3 | 100.0 | 1.00 | 0.25 |
| 27.657 | 0.0 | 100.0 | — | 0.24 |

^{a}μg/g dry weight

At a cut-off value of 13 ng/g dry weight ethyl oleate, sensitivity is 100%, specificity is 66.7%, positive predictive value is 0.91, and negative predictive value is 1.00. At a cut-off value of 77 ng/g dry weight ethyl oleate, sensitivity is 68.4%, specificity is 100%, positive predictive value is 1.00, and negative predictive value is 0.50.

Discussion:

The GC/MS/MS methodology identifies peaks by both retention time and mass weight, as compared to the less specific flame ionization detector (FID) method used in our previous study, which identifies eluting peaks solely in terms of retention times. At a specificity of 83%, the sensitivity provided by this methodology was 84%, compared with only 70% to detect heavy drinking (14 drinks/week prior to pregnancy) using the FID methodology; at a specificity of 100%, sensitivity was 68%, compared with 52%. Moreover, whereas the FID assays were most strongly related to reported drinking prior to conception, possibly due to the very low levels of later pregnancy drinking in that cohort, the GS/MS/MS assay results were most strongly related to second and third trimester drinking, which is the period during which the meconium forms in the fetus.

Increased concentrations of ethyl oleate were seen in meconium at 1.5 oz of alcohol (or 3 standard drinks) per drinking day or higher. Data from several U.S. studies have suggested that 5 drinks per occasion is the principal level of concern for prenatal alcohol exposure, because it is the critical dose most commonly associated with adverse neurobehavioral outcome. Thus, the ROC analysis suggests that the meconium assay can detect alcohol exposure at levels below which neurobehavioral deficits are generally found. Alternatively, however, the mothers in this cohort were shorter and weighed less than pregnant drinking women in the US. It is possible that peak blood alcohol concentration following a 3-drink binge in these smaller Cape Coloured women is equivalent to that of an average American woman following a 5-drink binge.

The correlation of average absolute ounces of alcohol per drinking day to ethyl oleate was not perfect. In some cases mothers who reported little or no drinking had somewhat higher FAEE concentrations, possibly due to some underreporting of drinking behavior, which is socially stigmatized. In other cases, mothers who reported high levels of drinking had FAEE concentrations that were similar to those who reported substantially less drinking. The meconium specimens that were collected were randomly sampled. FAEEs may accumulate unevenly in meconium reflecting the time of exposure, and the samples collected may not have formed at the time when the reported drinking behavior occurred. In addition, the genetic polymorphisms in alcohol dehydrogenase and acetaldehyde dehydrogenase in the mother may influence the synthesis of FAEEs. It is also of note that among the six women who reported abstinence, five had detectable ethyl oleate concentrations in their infants' meconium. Because ethanol is a byproduct of human metabolism, some baseline level of FAEEs is to be expected. In addition, there are alternative sources of exposure to ethanol, including elixirs, certain over-the-counter medications, and food additives. Thus, by contrast to the metabolites of drugs of abuse, the mere presence of FAEEs in meconium does not indicate ingestion of alcoholic beverages. While drugs of abuse have been shown not to influence FAEE levels, the degree to which maternal illness and medication use influence FAEE concentration has yet to be determined.

These studies have shown that ingestion of a given dose of alcohol over a short time period generates a greater peak blood alcohol concentration and greater neuronal and behavioral impairment than when the same dose is ingested gradually over several days. Based on these data, a biomarker of number of drinks per occasion might be more predictive of FAS/ARND than a marker of weekly average alcohol consumption. The association of FAEEs in meconium with alcohol dose per occasion shown in FIG. 1 suggests that pregnant women may be able to metabolize a low dose of alcohol relatively rapidly and that FAEE metabolites will be detected in the meconium primarily when alcohol ingestion has exceeded a certain threshold, making the assay particularly useful for identifying women at risk.

The availability of a reliable biomarker of alcohol consumption during pregnancy represents a major advance in the diagnosis and treatment of FAS and ARND. The diagnosis of FAS is difficult due to the extensive training required to identify the relevant craniofacial features, and no reliable behavioral profile has emerged for diagnosing ARND. FAS diagnosis is particularly difficult in the newborn; the facial features are most prominent in the school-age child. It is difficult to ascertain how much alcohol the mother has consumed during pregnancy due to the difficulty of recalling quantities and frequencies of alcohol intake. Moreover, the heavy stigma attached to pregnancy drinking makes obstetricians and midwives reluctant to inquire about drinking and pregnant women even more reluctant to reveal this information. The meconium assay can also facilitate the identification of alcohol-exposed infants, making it possible to intervene with these children earlier in development when interventions are often most effective. Alcohol-exposed children have fewer secondary disabilities if diagnosed before age 6 years. Laboratory experiments with prenatally-exposed rats have recently demonstrated the efficacy of at least one form of early intervention, suggesting that similar interventions may benefit humans. A reliable biomarker for alcohol would also improve reliability of measurement in research designed to assess the effects of prenatal alcohol exposure and facilitate statistical control for alcohol exposure in studies of pregnancy smoking, illicit drug use, and other sources of developmental risk. The meconium assay is also potentially useful for evaluating the effectiveness of interventions designed to reduce maternal drinking during pregnancy since it provides an objective criterion for evaluating the mother's compliance with the intervention protocol.

From the above description of the invention, those skilled in the art will perceive improvements, changes and modification. Such improvements, changes and modifications within the skill of the art are intended to be covered by the appended claims.

1. A diagnostic test for determining maternal alcohol consumption during pregnancy, comprising;
determining the level of at least one fatty acid ethyl ester in a bodily sample of a neonate; and
comparing the level of the at least one fatty acid ethyl ester in the bodily sample with at least one predetermined value;
wherein such comparison provides information for determining maternal alcohol consumption during pregnancy.

2. The diagnostic test of claim 1 wherein the at least one predetermined value is a single normalized value or a range of normalized values and is based on fatty acid ethyl ester levels in comparable bodily samples from the general population or a select population of neonate subjects.

3. The diagnostic test of claim 1 wherein the at least one predetermined value is a single value or a range of representative values and is based on the fatty acid ethyl ester levels in the comparable bodily samples from the general population or a select population of neonate subjects.

4. The diagnostic test of claim 1 wherein the at least one predetermined value is a plurality of fatty acid ethyl ester level ranges that are based on the fatty acid ethyl ester levels in comparable bodily samples from the general population or a select population of neonate subjects, and
said comparing step comprises determining in which of said plurality of predetermined fatty acid ethyl ester level ranges the neonate's fatty acid ethyl ester level falls.

5. The diagnostic test of claim 1 wherein the at least one fatty acid ethyl ester has the general formula CₙH₂ₙ₋ₓO₂, where n is an integer greater than 12 and x is selected from group consisting of 0, 1, 2, 3, and 4.

6. The diagnostic test of claim 1 wherein the at least one fatty acid ethyl ester is selected from the group consisting of ethyl palmitate, ethyl oleate, and ethyl linoleate.

7. The diagnostic test of claim 1 wherein the level of the at least one fatty acid ethyl ester is determined by isolating the at least one fatty acid ethyl ester from the bodily sample and detecting the isolated fatty acid ethyl ester by mass spectrometry.

8. The diagnostic test of claim 7 wherein the at least one fatty acid ethyl ester is isolated from the bodily sample by extracting the at least one fatty acid ester from the bodily sample and isolating the extracted fatty acid ethyl ester using chromatographic separation.

9. The diagnostic test of claim 7 wherein the isolated fatty acid ethyl ester is detected by tandem mass spectrometry.

10. The diagnostic test of claim 1 wherein the level of the at least one fatty acid ethyl ester is determined by a diagnostic assay.

11. The diagnostic test of clam 9 wherein the diagnostic assay is selected from the group consisting of colorimetric assays and immunoassays.

12. The diagnostic test of claim 1 wherein the bodily sample comprises meconium.

13. The diagnostic test of claim 1 wherein the comparison provides information for average alcohol per drinking day.

14. A diagnostic test for characterizing a neonate's risk of developing or having fetal alcohol syndrome and/or an alcohol-related neurodevelopmental disorder, comprising
determining the level of the at least one fatty acid ethyl ester in a bodily sample of a neonate; and
comparing the level of the at least one fatty acid ethyl ester in the bodily sample of the neonate with at least one predetermined value;
wherein such comparison provides information for characterizing the neonate's risk of developing or having fetal alcohol syndrome and/or alcohol-related neurodevelopmental disorder.

15. The diagnostic test of claim 14 wherein the at least one predetermined value is a single normalized value or a range of normalized values and is based on fatty acid ethyl ester levels in comparable bodily samples from the general population or a select population of neonate subjects.

16. The diagnostic test of claim 14 wherein the at least one predetermined value is a single value or a range of representative values and is based on the fatty acid ethyl ester levels in the comparable bodily samples from the general population or a select population of neonate subjects.

17. The diagnostic test of claim 14 wherein the at least one predetermined value is a plurality of fatty acid ethyl ester level ranges that are based on the fatty acid ethyl ester levels in comparable bodily samples from the general population or a select population of neonate subjects; and
said comparing step comprises determining in which of said plurality of predetermined fatty acid ethyl ester level ranges the neonate's fatty acid ethyl ester level falls.

18. The diagnostic test of claim 14 wherein the at least one fatty acid ethyl ester has the general formula CₙH₂ₙ₋ₓO₂, where n is an integer greater than 12 and x is selected from group consisting of 0, 1, 2, 3, and 4.

19. The diagnostic test of claim 14 wherein the at least one fatty acid ethyl ester is selected from the group consisting of ethyl palmitate, ethyl oleate, and ethyl linoleate.

20. The diagnostic test of claim 14 wherein the level of the at least one fatty acid ethyl ester is determined by isolating the at least one fatty acid ethyl ester from the bodily sample and detecting the isolated fatty acid ethyl ester by mass spectrometry.

21. The diagnostic test of claim 14 wherein the at least one fatty acid ethyl ester is isolated from the bodily sample by extracting the at least one fatty acid ester from the bodily sample and isolating the extracted fatty acid ethyl ester using chromatographic separation.

22. The diagnostic test of claim 14 wherein the isolated fatty acid ethyl ester is detected by tandem mass spectrometry.

23. The diagnostic test of claim 14 wherein the level of the at least one fatty acid ethyl ester is determined by a diagnostic assay.

24. The diagnostic test of claim 23 wherein the diagnostic assay is selected from the group consisting of calorimetric assays and immunoassays.

25. The diagnostic test of claim 14 wherein the bodily sample comprises meconium.

26. The diagnostic test of claim 14 wherein the comparison provides information for average alcohol per drinking day.

27. A diagnostic test for determining maternal alcohol consumption during pregnancy, comprising;
isolating fatty acid ethyl esters from a bodily sample of a neonate; and
determining the level of at least one of ethyl linoleate, ethyl palmitate, and/or ethyl oleate in the isolated fatty acid ethyl esters,
comparing the level of the of at least one of ethyl linoleate, ethyl palmitate, and/or ethyl oleate with at least one predetermined value;
wherein such comparison provide information for determining maternal alcohol consumption during pregnancy.

28. The diagnostic test of claim 27 wherein the level of ethyl linoleate, ethyl palmitate, and/or ethyl oleate is determined using tandem mass spectrometry.

30. The diagnostic test of claim 27 wherein the bodily sample comprises meconium.

31. The diagnostic test of claim 30 wherein the comparison provides information for average alcohol per drinking day.

32. A kit comprising, a means for at least partially isolating fatty acid ethyl esters from a bodily sample; and an assay for determining the level of at least one of ethyl linoleate, ethyl palmitate, and/or ethyl oleate in the isolated fatty acid ethyl esters.
